(19) United States
(12) Patent Application Publication
(10) Pub. No.: US 2021/0355066 A1
(43) Pub. Date: Nov. 18, 2021

(54) CB-0406 choline salt

(71) Applicants: CymaBay Therapeutics, Inc., Newark, CA (US); DiaTex, Inc., San Antonio, TX (US)

(72) Inventors: Stephan X.M. Boerrigter, West Lafayette, IN (US); Charles A. McWherter, Oakland, CA (US); Robert L. Martin, San Ramon, CA (US); Jennifer L. Nelson, Kokomo, IN (US); Jiangao Song, Sunnyvale, CA (US)

(73) Assignees: CymaBay Therapeutics, Inc., Newark, CA (US); DiaTex, Inc., San Antonio, TX (US)

(21) Appl. No.: 17/318,698

(22) Filed: May 12, 2021

Related U.S. Application Data
(60) Provisional application No. 63/026,239, filed on May 18, 2020.

(51) Int. Cl. C07C 59/72; C07C 215/40

(52) U.S. Cl. CPC C07C 59/72; C07B 2200/13; C07C 215/40

(57) ABSTRACT

CB-0406 choline salt, especially in crystalline form and as an ansolvate, methods of preparing it, compositions containing it, and its pharmaceutical uses.

### CROSS-REFERENCE TO RELATED APPLICATION

This application claims the benefit under 35 USC 119(e) of Application No. 63/026,239, “CB-0406 choline salt”, filed 18 May 2020, the entire content of which is incorporated into this application by reference.

### FIELD OF THE INVENTION

This invention relates to the choline salt of CB-0406.

### DESCRIPTION OF THE RELATED ART

CB-0406

CB-0406 is the compound having the IUPAC name of (2R)-2-(4-chlorophenyl)-2-[3-(trifluoromethyl)phenoxy]acetic acid, sometimes also given as (R)-2-(4-chlorophenyl)-2-[3-(trifluoromethyl)phenoxy]acetic acid. Other names that are or have been used for CB-0406 are (−)-CPTA, and arhalofenic acid or arhalofenate acid, since it is the underlying acid of the compound arhalofenate [INN/USAN; (−)-2-(acetylamino)ethyl (2R)-2-(4-chlorophenyl)-2-[3-(trifluoromethyl)-phenoxy]acetate; MBX-0102, CB-0102]. CB-0406 is the active metabolite of arhalofenate [see, for example, McWherter et al., “Arhalofenate acid inhibits monosodium urate crystal-induced inflammatory responses through activation of AMP-activated protein kinase (AMPK) signaling”, Arthritis Res. Ther., vol. 20, 204 (2018), https://doi.org/10.1186/s13075-018-1699-4]. U.S. Pat. No. 6,262,118 discloses the use of arhalofenate, CB-0406, and related compounds for the treatment of insulin resistance, type 2 diabetes, and hyperlipidemia, and U.S. Pat. No. 6,613,802 adds the treatment of hyperuricemia to that list. Those patents explain that arhalofenate and related compounds avoid certain drug-drug interactions seen with the racemate, such as with sulfonylureas, NSAIDs, and the anticoagulant warfarin, an interaction believed to be mediated by inhibition of certain cytochrome P450 enzymes, particularly CYP 2C9; and demonstrate that CB-0406 was approximately 20-fold less active as an inhibitor of CYP 2C9 than its (S)-enantiomer in the tolbutamide hydroxylation assay. U.S. Pat. Nos. 9,023,856 and 9,060,987, for example, disclose the treatment of hyperuricemia and gout, including gout flares, with arhalofenate, CB-0406 and its salts, and related compounds.

U.S. Pat. No. 6,262,118 discloses a synthesis of CB-0406 by resolution of its racemate with (−)-cinchonidine, thereby isolating the (−)-cinchonidine salt of CB-0406. U.S. Pat. No. 7,199,259 discloses a synthesis of CB-0406 by resolution with various agents, in particular (1R,2R)-2-amino-1-(4-nitrophenyl)propane-1,3-diol [CAF D base], thereby isolating the CAF D base salt of CB-0406; and U.S. Pat. No. 7,432,394 discloses a synthesis of CB-0406 by resolution of its racemate with a variety of chiral aralkylamines, in particular (S)-1-(2-naphthyl)ethylamine, thereby isolating the (S)-1-(2-naphthyl)ethylamine salt of CB-0406. Others, e.g. U.S. Pat. Nos. 7,714,131 and 8,541,614, disclose stereoselective syntheses, typically considering CB-0406 as an intermediate to arhalofenate.

U.S. Pat. No. 9,023,856, for example, says the following about salts of CB-0406:
“Pharmaceutically acceptable salt” includes pharmaceutically acceptable acid addition salts and pharmaceutically acceptable base addition salts and includes both solvated and unsolvated forms. Representative non-limiting lists of pharmaceutically acceptable salts can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, both of which are hereby incorporated by reference herein.
“Pharmaceutically acceptable base addition salt” refers to salts prepared from the addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

The disclosures of the documents referred to in this application are incorporated into this application by reference.

### SUMMARY OF THE INVENTION

In a first aspect, this invention is CB-0406 choline salt. In particular, this aspect is crystalline CB-0406 choline salt, CB-0406 choline salt ansolvate, and especially crystalline CB-0406 choline salt ansolvate.

In a second aspect, this invention is methods of preparing the CB-0406 choline salt of the first aspect of this invention.

In a third aspect, this invention is pharmaceutical compositions, especially oral pharmaceutical compositions, containing the CB-0406 choline salt of the first aspect of this invention.

In a fourth aspect, this invention is pharmaceutical uses of the CB-0406 choline salt of the first aspect of this invention in the treatment of conditions for which arhalofenate, or CB-0406 and its salts, are indicated.

Preferred embodiments of this invention are characterized by the specification and by the features of claims 1 to 13 of this application as filed.

### BRIEF DESCRIPTION OF THE DRAWINGS

FIG. 1 is a differential scanning calorimetry (DSC) thermogram of CB-0406 choline salt.

FIG. 2 is a thermogravimetric analysis (TGA) thermogram of CB-0406 choline salt.

FIG. 3 is an X-ray powder diffraction (XRPD) pattern of CB-0406 choline salt.

### DETAILED DESCRIPTION OF THE INVENTION

### Definitions

“CB-0406” is described in the section “CB-0406” in the DESCRIPTION OF THE RELATED ART.

“Choline” has the IUPAC name 2-hydroxy-N,N,N-trimethylethan-1-aminium; and is sometimes also referred to as (2-hydroxyethyl)trimethylammonium. It is the cation of the base choline hydroxide, 2-hydroxy-N,N,N-trimethylethan-1-aminium hydroxide (choline base; usually a viscous, strongly alkaline liquid, though reportedly crystallizable; typically available as a ˜45% solution in water or methanol), and the salts choline chloride, 2-hydroxy-N,N,N-trimethylethan-1-aminium chloride, and other salts.

“CB-0406 choline salt” is the 1:1 salt formed between (2R)-2-(4-chlorophenyl)-2-[3-(trifluoromethyl)phenoxy]acetic acid and 2-hydroxy-N,N,N-trimethylethan-1-aminium hydroxide. It may be named 2-hydroxy-N,N,N-trimethylethan-1-aminium (2R)-2-(4-chlorophenyl)-2-[3-(trifluoromethyl)phenoxy]acetate. “Crystalline CB-0406 choline salt” is a crystalline solid form of CB-0406 choline salt. The “ansolvate” of CB-0406 choline salt is a form of CB-0406 choline salt that is free of solvents associated with the salt, including water; but bulk material may contain small amounts of one or more solvents, such as the solvents used in its synthesis. The “crystalline ansolvate” of CB-0406 choline salt is a crystalline form of CB-0406 choline salt that is free of solvents of crystallization associated with the salt, including water; but bulk material may contain small amounts of one or more solvents, such as the solvents used in its synthesis or crystallization.

“Characterization” refers to obtaining data that may be used to identify a solid form of a compound; for example, whether the solid form is amorphous or crystalline and whether it is unsolvated or solvated. The process by which solid forms are characterized involves analyzing data collected on the forms to allow a person of ordinary skill in the art to distinguish one solid form from other solid forms containing the same material. Chemical identity of solid forms can often be determined with solution-state techniques such as ¹³C nuclear magnetic resonance (NMR) spectroscopy or ¹H NMR. While these may help identify a material, and a solvent molecule for a solvate, such solution-state techniques themselves do not provide information about the solid state. There are, however, solid-state analytical techniques that can be used to provide information about solid-state structure and differentiate among solid forms such as polymorphs, including single crystal X-ray diffraction, XRPD, solid state NMR, infrared and Raman spectroscopy, and thermal techniques such as DSC, TGA, melting point, and hot-stage microscopy.

An XRPD pattern is an x-y graph with diffraction angle 2θ (typically in degrees, °) on the x-axis and intensity on the y-axis. The peaks within this pattern may be used to characterize a crystalline solid form. As with any data measurement, there is variability in XRPD data. The data are frequently represented solely by the diffraction angle of the peaks rather than including the intensity of the peaks because peak intensity can be particularly sensitive to sample preparation, for example, because of particle morphology and size, moisture content, solvent content, and preferred orientation effects, so samples of the same material prepared under different conditions may yield slightly different XRPD patterns; and this variability is usually greater than the variability in diffraction angles. Diffraction angle variability may also be sensitive to sample preparation. Other, but less significant, sources of diffraction angle variability come from instrument parameters and processing of the raw X-ray data: different instruments operate using different parameters and these may lead to slightly different XRPD patterns even from the same solid form, and similarly different software packages process X-ray data differently and this also leads to variability. These and other sources of variability are known to those of ordinary skill in the pharmaceutical arts. Due to such sources of variability, it is usual to assign a variability of ±0.2° to diffraction angles (20) in XRPD patterns, especially when using those angles for characterization of a solid form.

To characterize a solid form of a compound a person of ordinary skill in the art may, for example, collect XRPD data on solid forms of the compound and compare the XRPD peaks of the forms. When only two solid forms, I and II, are compared and the Form I XRPD pattern shows a peak at an angle where no peaks appear in the Form II XRPD pattern, then for that compound that peak distinguishes Form I from Form II and further acts to characterize Form I. The collection of peaks that distinguish Form I from the other known forms is a collection of peaks that may be used to characterize Form I. Additional peaks could also be used, but are not necessary, to characterize the form, up to and including an entire XRPD pattern; however, a subset of that data may, and typically is, used to characterize the form. A person of ordinary skill in the art will recognize that there are often multiple ways, including multiple ways using the same technique, to characterize solid forms.

“Comprising” or “containing” and their grammatical variants are words of inclusion and not of limitation and mean to specify the presence of stated components, groups, steps, and the like but not to exclude the presence or addition of other components, groups, steps, and the like. Thus “comprising” does not mean “consisting of”, “consisting substantially of”, or “consisting only of”; and, for example, a formulation “comprising” a compound must contain that compound but also may contain other active ingredients and/or excipients.

CB-0406 choline salt has been characterized using DSC, TGA, XRPD, and solution ¹H NMR. The solubility of CB-0406 choline salt has been measured in simulated intestinal fluid without pancreatin.

Preparation of CB-0406 Choline Salt

CB-0406 (62.1 mg) and one molar equivalent of choline base (21.1 mg) were dissolved in 83/17 v/v MeOH/H₂O (˜1.2 mL). The solution was evaporated to dryness and then vacuum-dried at ambient temperature for one day. Anhydrous methyl tert-butyl ether (MTBE) (˜0.5 mL) was added, the sample was sonicated briefly, and then stirred for one day at ambient temperature. The solids were isolated by vacuum filtration, and the wet cake was washed twice with ˜0.5 mL of anhydrous heptane and vacuum dried to give CB-0406 choline salt.

Characterization of CB-0406 Choline Salt

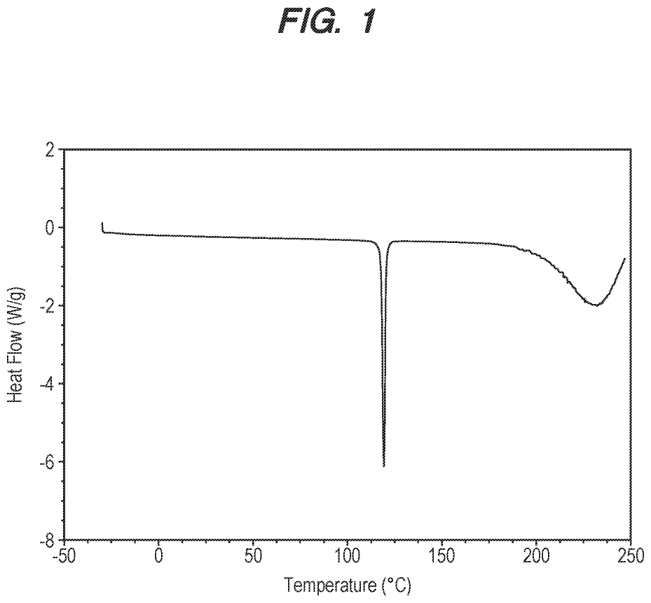

A DSC analysis of CB-0406 choline salt was performed using a TA Instruments Q2000 differential scanning calorimeter. Temperature calibration was performed using NIST-traceable indium metal. The sample, 1.74 mg, was placed into an aluminum DSC pan, covered with a lid which was crimped at the beginning of the run, and the weight was accurately recorded. A weighed aluminum pan configured as the sample pan was placed on the reference side of the cell. The sample cell was heated from −30° C. to 250° C. at 10° C./minute. As shown in FIG. 1, DSC showed a steep initial endotherm with onset at about 118° C. and peak (86.2 J/g) at 119.1° C., with a broad endotherm peaking at around 230° C. The variability of DSC data is affected by sample preparation and particularly by heating rate.

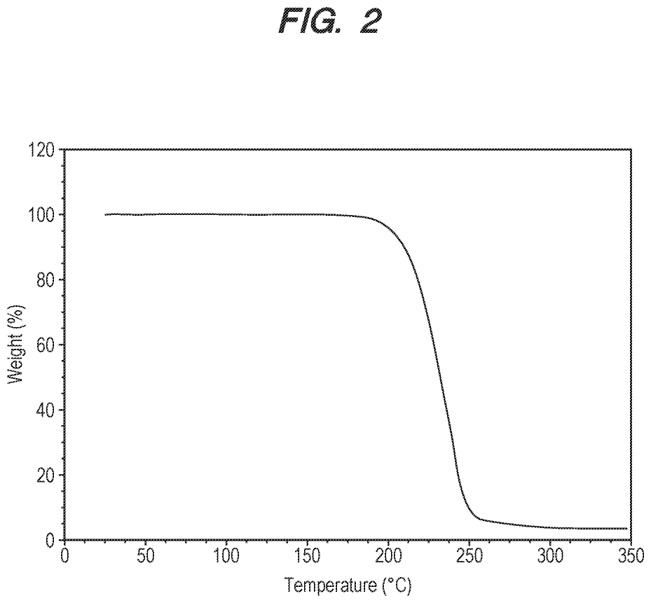

A TG analysis of CB-0406 choline salt was performed using a TA Instruments 2950 thermogravimetric analyzer. Temperature calibration was performed using nickel and Alumel™ The sample, 7.175 mg, was placed in an aluminum pan and inserted into the TG furnace. The furnace was heated under a nitrogen purge. The sample cell was heated from ambient temperature to 350° C. at 10° C./minute. As shown in FIG. 2, TGA showed a negligible loss in weight (0.1%) between 30° C. and 140° C., and a steepening loss starting at about 200° C. As with DSC data, the variability of TGA data is affected by sample preparation and particularly by heating rate.

The XRPD pattern of CB-0406 choline salt was collected with a PANalytical X'Pert PRO MPD diffractometer using an incident beam of Cu radiation produced using an Optix long, fine-focus source at 45 kV and 40 mA, with a 0.5° divergence slit before the mirror. An elliptically graded multilayer mirror was used to focus Cu Kα X-rays through the specimen and onto the detector. Prior to the analysis, a silicon specimen (NIST SRM 640d) was analyzed to verify the observed position of the Si 111 peak is consistent with the NIST-certified position. A specimen of the sample was sandwiched between 3 μm thick films and analyzed in transmission geometry. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 2.2b. The scan range was (1.00-39.99)° 20, with a scan speed of 3.3°/minute (step size 0.017° 20).

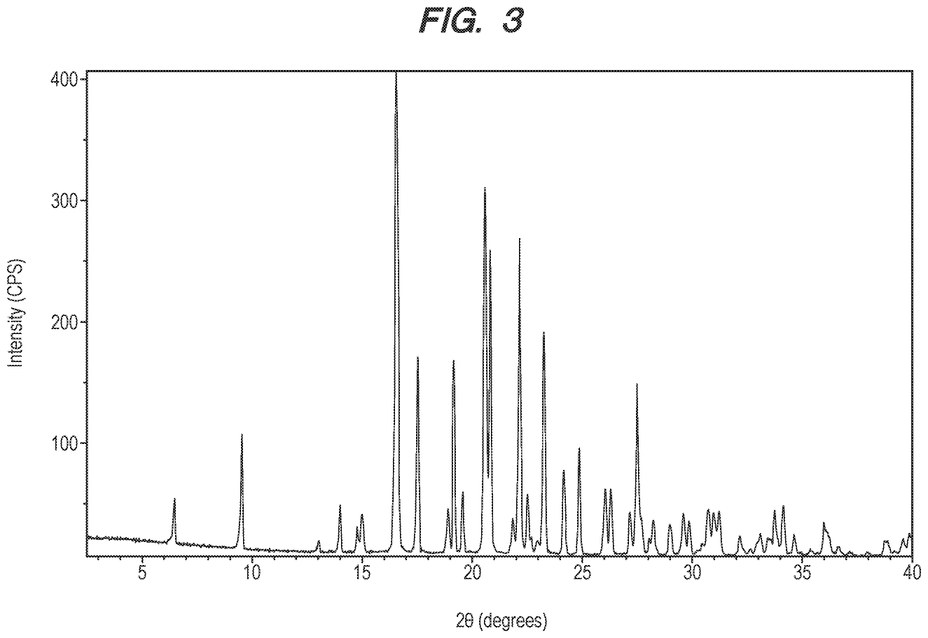

The XRPD pattern is shown in FIG. 3. The location of the peaks along the horizontal axis was automatically determined using proprietary software (PatternMatch v.3.0.4) and rounded to two decimal places. Peaks in diffraction intensity, with the intensity in parentheses as a percentage of the maximum recorded intensity (the intensity of the peak at 16.55°), were determined from the XRPD pattern of FIG. 3 at 6.49° (13), 9.53° (27), 13.00° (5), 13.99° (12), 14.77° (8), 14.97° (10), 16.55° (100), 17.52° (43), 18.89° (11), 19.15° (42), 19.57° (15), 20.56° (78), 20.81° (65), 21.83° (9), 22.13° (66), 22.50° (15), 22.66° (6), 22.96° (5), 23.23° (47), 24.13° (19), 24.82° (24), 26.02° (15), 26.27° (15), 27.14° (11), 27.46° (37), 28.03° (5), 28.19° (9), 28.95° (8), 29.56° (11), and 29.81° (9).

Prominent peaks usable for characterization may be selected from this list, such as those at having intensities greater than 15% of the maximum recorded intensity (the intensity of the peak at) 16.55°, i.e., peaks at 6.5°, 9.5°, 16.6°, 17.5°, 19.2°, 20.6°, 20.8°, 22.1°, 23.2°, 24.1°, 24.8°, 26.0°, 26.3°, and 27.5°; figures here are rounded to only one decimal place because of the assumed ±0.2° variability in 20, and the peak at 6.5° is included despite an intensity of 13% because of its low diffraction angle. Of these, low diffraction angle and high intensity peaks are of greatest interest, such as the peaks at 6.5°, 9.5°, 16.6°, 20.6°, 20.8°, and 22.1° 20. An XRPD pattern “substantially similar” to the pattern shown in FIG. 3 will exhibit at least four of the peaks listed in the preceding sentence to within ±0.2° in 20, though not necessarily at the intensities listed in the previous paragraph.

A solution ¹H NMR spectrum of CB-0406 choline salt was acquired with a Varian UNITY/NOVA-400 spectrometer. The sample was prepared by dissolving a small amount of CB-0406 choline salt, prepared as described previously, in DMSO-d₆ containing tetramethylsilane. The spectrum of CB-0406 choline salt was consistent with the presence of deprotonated CB-0406 to choline in about a 1:1 ratio, with a trace of MTBE.

CB-0406 choline salt was determined to have a solubility >200 mg/mL in simulated intestinal fluid without pancreatin.

Pharmaceutical Formulations

CB-0406 choline salt is expected to be of pharmaceutical utility because of its ability to be produced in crystalline form, with a higher melting point than crystalline CB-0406 (i.e. ˜118° C. for CB-0406 choline salt, ˜99° C. for CB-0406), and with good stability to thermal stress. It also has high solubility in simulated intestinal fluid (at least ˜60-fold greater than that of CB-0406), leading to expected high oral bioavailability. Though it is expected to be useful in formulations other than oral formulations because of its desirable pharmaceutical properties, it is expected to be of particular value in oral formulations. Suitable formulations for various methods of administration may be found, for example, in “Remington: The Science and Practice of Pharmacy”, 20th ed., Gennaro, ed., Lippincott Williams & Wilkins, Philadelphia, Pa., U.S.A. Because CB-0406 choline salt is soluble and therefore orally available, typical formulations will be oral, and typical dosage forms will be tablets or capsules for oral administration. In addition to an effective amount of the CB-0406 choline salt, the compositions may contain one or more suitable pharmaceutically-acceptable excipients, including fillers, stabilizers such as antioxidants, disintegrating agents, and processing aids such as binders, glidants, and lubricants, which facilitate processing of the CB-0406 choline salt into preparations which can be used pharmaceutically. “Pharmaceutically acceptable excipient” refers to an excipient or mixture of excipients which does not interfere with the effectiveness of the biological activity of the active compound(s) and which is not toxic or otherwise undesirable to the subject to which it is administered. For solid compositions, conventional excipients include, for example, pharmaceutical grades of mannitol, lactose, starch, magnesium stearate, sodium saccharin, talc, cellulose, glucose, sucrose, magnesium carbonate, and the like.

Pharmaceutical Uses

CB-0406 choline salt, as a salt of CB-0406, is expected to be pharmaceutically useful in the treatment of all conditions for which arhalofenate, or CB-0406 and its salts, are indicated. It is thus expected to be useful for the treatment of insulin resistance, type 2 diabetes, hyperlipidemia, and hyperuricemia, as described for example in U.S. Pat. Nos. 6,262,118 and 6,613,802; and for the treatment of hyperuricemia and gout, including gout flares, as described for example in U.S. Pat. Nos. 9,023,856 and 9,060,987.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

1. A compound that is 2-hydroxy-N,N,N-trimethylethan-1-aminium (2R)-2-(4-chlorophenyl)-2-[3-(trifluoromethyl)phenoxy]acetate.

2. The compound of claim 1 in crystalline form.

3.-13. (canceled)

14. The compound of claim 1 that is an ansolvate.

15. The compound of claim 14 in crystalline form.

16. The compound of claim 1 characterized by at least one of (a), (b), or (c):
(a) an endothermic peak at (119±2) ° C. as measured by differential scanning calorimetry;
(b) a substantial absence of weight loss below 140° C. as measured by thermogravimetric analysis;
(c) at least one X-ray powder diffraction peak (Cu Kα radiation) selected from 6.5°, 9.5°, 16.6°, 17.5°, 19.2°, 20.6°, 20.8°, 22.1°, 23.2°, 24.1°, 24.8°, 26.0°, 26.3°, or 27.5° (each ±0.2°) 2θ.

17. The compound of claim 16 characterized by an endothermic peak at (119±2) ° C. as measured by differential scanning calorimetry.

18. The compound of claim 17 characterized by a substantial absence of thermal events at temperatures below the endothermic peak at (119±2) ° C. as measured by differential scanning calorimetry.

19. The compound of claim 16 characterized by a substantial absence of weight loss below 140° C. as measured by thermogravimetric analysis.

20. The compound of claim 16 characterized by at least one X-ray powder diffraction peak (Cu Kα radiation) selected from 6.5°, 9.5°, 16.6°, 17.5°, 19.2°, 20.6°, 20.8°, 22.1°, 23.2°, 24.1°, 24.8°, 26.0°, 26.3°, or 27.5° (each ±0.2°) 2θ.

21. The compound of claim 20 characterized by at least two X-ray powder diffraction peaks (Cu Kα radiation) selected from 6.5°, 9.5°, 16.6°, 17.5°, 19.2°, 20.6°, 20.8°, 22.1°, 23.2°, 24.1°, 24.8°, 26.0°, 26.3°, or 27.5° (each ±0.2°) 2θ.

22. The compound of claim 21 characterized by at least one, preferably at least two, especially at least three X-ray powder diffraction peaks (Cu Kα radiation) selected from 6.5°, 9.5°, 16.6°, 17.5°, 19.2°, 20.6°, 20.8°, 22.1°, 23.2°, 24.1°, 24.8°, 26.0°, 26.3°, or 27.5° (each ±0.2°) 2θ.

23. The compound of claim 20 characterized by an X-ray powder diffraction peak (Cu Kα radiation) at (16.6±0.2)° 2θ.

24. The compound of claim 23 characterized by an X-ray powder diffraction pattern (Cu Kα radiation) substantially similar to that of FIG. 3.

25. A method of preparing the compound of claim 1 comprising reacting (2R)-2-(4-chlorophenyl)-2-[3-(trifluoromethyl)phenoxy]acetic acid with 2-hydroxy-N,N,N-trimethylethan-1-aminium hydroxide in methanol/water.

26. The method of claim 25 further comprising drying the 2-hydroxy-N,N,N-trimethylethan-1-aminium (2R)-2-(4-chlorophenyl)-2-[3-(trifluoromethyl)phenoxy]-acetate by removal of the methanol/water, followed by sonication of the solids in anhydrous methyl tert-butyl ether and isolation of the 2-hydroxy-N,N,N-trimethylethan-1-aminium (2R)-2-(4-chlorophenyl)-2-[3-(trifluoromethyl)phenoxy]acetate by filtration.

27. A solid pharmaceutical formulation comprising the compound of claim 1 and at least one pharmaceutically acceptable excipient.

28. A method of treating a condition for which administration of arhalofenate, or of (2R)-2-(4-chlorophenyl)-2-[3-(trifluoromethyl)phenoxy]acetic acid or a salt thereof, is indicated, comprising administration of a therapeutically effective amount of the compound of claim 1.
